(19) United States
(12) Patent Application Publication
(10) Pub. No.: US 2018/0168214 A1
(43) Pub. Date: Jun. 21, 2018

(54) MARINE PEPTIDES AND MUSCLE HEALTH

(71) Applicant: Firmenich SA, Geneva 8 (CH)

(72) Inventor: Einar LIED, Bergen (NO)

(73) Assignee: Firmenich SA, Geneva 8 (CH)

(21) Appl. No.: 15/736,851

(22) PCT Filed: Jun. 29, 2016
(86) PCT No.: PCT/EP2016/065200

§ 371 Date: Dec. 15, 2017

Related U.S. Application Data
(60) Provisional application No. 62/187,212, filed on Jun. 30, 2015.

(51) Int. Cl. A23L 33/18; A23J 1/04; A23J 3/04; A23K 10/22; A61K 35/60; A61P 21/00

(52) U.S. Cl. CPC A23L 33/18; A23J 1/04; A23J 3/04; A23K 10/22; A23V 2250/543; A61P 21/00; A23V 2002/00; A23V 2200/316; A61K 35/60

(57) ABSTRACT

Provided herein are marine peptides for use in increasing muscle mass and growth and enhancing muscle recovery. The peptides provided herein are useful as nutritional supplements, food, functional foods, and as pharmaceutical formulations.

### FIELD

Provided herein are fish products and by-products and their use in health, food, nutrition and medical treatment.

### BACKGROUND

Protein hydrolysates have shown some health benefits. Data show that protein ingestion before a meal, when consumed with carbohydrate, reduces postprandial blood glucose; the blood sugar lowering effects have in particular been linked to dairy proteins such as whey proteins. A study comparing whey protein with whey protein hydrolysate showed that whey protein consumed before a meal reduces food intake, postprandial blood glucose and insulin, and the ratio of cumulative blood glucose to insulin area under the curves (AUCs) in a dose-dependent manner. In contrast whey protein hydrolysate did not contribute to blood glucose control neither by insulin-dependent nor insulin-independent mechanisms.

Proteins are important for example in muscle metabolism and it is desirable to increase lean body mass including muscle for example in the fields of sports and physical exercise. Muscle breakdown and soreness occurs with exercise. Hence, it is desirable to provide nutritional sources of proteins that may increase muscle mass and strength as well as enhancing physical performance and recovery from exercise.

### SUMMARY

Provided herein is a method of increasing muscle growth in an animal comprising administering to an animal in need of muscle growth an effective amount of a marine peptide having the peptide molecular weight profile (Profile I):

| Peptide (Molecular Weight (Da) | Amount (Dried Weight Bases) |
|---|---|
| >20 KDa | 0-5% |
| 15 KDa-20 KDa | 0-5% |
| 10 KDa-15 KDa | 0-5% |
| 8 KDa-10 KDa | 0-5% |
| 6 KDa-8 KDa | 0-5% |
| 4 KDa-6 KDa | 0-10% |
| 2 KDa-4 KDa | 0-15% |
| 1 KDa-2 KDa | 0-50% |
| 0.5 KDa-1 KDa | 0-50% |
| 0.2 KDa-0.5 KDa | 0-75% |
| <0.2 KDa | 0-75%. |

The total amount of the peptides is 100% (dried weight basis).

### DETAILED DESCRIPTION

For the descriptions herein and the appended claims, the singular forms “a”, “an” and “the” include plural referents unless the context clearly indicates otherwise. Thus, for example, reference to “a compound” refers to more than one compound. Also, the use of “or” means “and/or” unless stated otherwise. Similarly, “comprise,” “comprises,” “comprising” “include,” “includes,” and “including” are interchangeable and not intended to be limiting.

For the descriptions herein and the appended claims, the singular forms “a”, “an” and “the” include plural referents unless the context clearly indicates otherwise. Thus, for example, reference to “a compound” refers to more than one compound. Also, the use of “or” means “and/or” unless stated otherwise. Similarly, “comprise,” “comprises,” “comprising” “include,” “includes,” and “including” are interchangeable and not intended to be limiting.

In one embodiment animal is a mammal.

In one embodiment, the mammal is a human.

In one embodiment the animal is in need of muscle growth, increased muscle mass, and muscle recovery.

In one embodiment, the marine peptide of Profile I is administered to an animal in an amount of about 20 mg of the marine peptide of Profile I per one Kg of body weight of the animal.

In one embodiment, the marine peptide of Profile I is administered to an animal in an amount of about 40 mg of marine peptide of Profile I per one Kg of body weight of the animal.

In one embodiment, the marine peptide of Profile I is administered to an animal in an amount of from about 5 mg to about 40 mg per Kg bodyweight, more particularly from about 10 mg to about 30 mg per Kg bodyweight and even more particularly about 20 mg per Kg bodyweight of the animal. In a more particular embodiment the marine peptide of Profile I is provided in an amount of about 10 mg per Kg bodyweight of the animal.

In one embodiment a dietary supplement is provided comprising a marine peptide of Profile I in an amount sufficient for the maintenance, in an animal, of muscle health.

In one embodiment a dietary supplement is provided comprising a marine peptide of Profile I in an amount sufficient for muscle recovery after exercise.

In one embodiment a dietary supplement is provided comprising a marine peptide of Profile I in an amount sufficient to increase muscle mass.

In one embodiment a food is provided comprising a marine peptide of Profile I in an amount sufficient for the maintenance, in an animal, of muscle health.

In one embodiment a food is provided comprising a marine peptide of Profile I in an amount sufficient for muscle recovery after exercise.

In one embodiment a food is provided comprising a marine peptide of Profile I in an amount sufficient to increase muscle mass.

In one embodiment a food is provided comprising a marine peptide of Profile I described herein in an amount sufficient for the maintenance, of muscle health.

In one embodiment a method is provided comprising administering a food comprising a marine peptide of Profile I to manage the levels of nitrogen in an animal.

In one embodiment, the marine peptide of Profile I provided herein is delivered to a human in an effective amount to increase muscle mass, increase muscle strength, increase physical performance and/or to enhance muscle recovery

In one embodiment, the marine peptide of Profile I provided herein is delivered to a human at meal time.

In another embodiment, the marine peptide of Profile I provided herein is administered to a human before exercise, during exercise, after exercise, or a combination thereof.

In another embodiment the marine peptide of Profile I provided herein is delivered to a human in an amount effective to maintain muscle health.

In one embodiment, the marine peptide of Profile I is administered to a human in an amount of from about 1 gram up to about 10 grams, particularly from about 2 grams to about 10 grams, more particularly from about 2 grams up to about 5 grams and more particularly from about 2 grams to about 3 grams.

Provided herein is a method comprising administering to a human an effective amount of a marine peptide of Profile I wherein the marine peptide of Profile I is administered in a at least one unit dose wherein the unit dose is selected from the group consisting of 25 mg, 50 mg, 75 mg, 100 mg, 125 mg, 150 mg, 175 mg, 200 mg, 225 mg, 250 mg, 275 mg, 300 mg, 325 mg, 350 mg, 400 mg, 425 mg, 450 mg, 475 mg, 500 mg, 1000 mg, 1500 mg 2000 mg, 2500 mg and 3000 mg of marine peptide of Profile I.

In some embodiments, the animal to be treated can be administered at least one unit dose per day. In some embodiments, the dosage forms can be taken in a single application or multiple applications per day. For example, if four capsules are taken daily, each capsule comprising about 500 mg marine peptide of Profile I, then all four capsules could be taken once daily, or 2 capsules could be taken twice daily, or 1 capsule could be taken every 6 hours. In some embodiments, the marine peptide of Profile I is administered in a single dosage form, i.e., a dosage form, or in two or more dosage forms. As used herein, “dosage form” refers to the physical form for the route of administration. The term “dosage form” can refer to any traditionally used or medically accepted administrative forms, such as oral administrative forms, intravenous administrative forms, or intraperitoneal administrative forms. In some embodiments, the marine peptide of Profile I is administered in a single dose, i.e., a unit dose. As used herein, a “unit dose” refers to an amount of marine peptide of Profile I administered to an animal in a single dose, e.g., in a gel capsule. The term “unit dose” can also refer to a single unit of pharmaceutically suitable solid, liquid, syrup, beverage, or food item, that is administered within a short period of time, e.g., within about 1 minute, 2 minutes, 3 minutes, 5 minutes, 10 minutes, 20 minutes, or 30 minutes.

A marine peptide of Profile I may be obtained for example, but not limited to fish, marine algae, crustaceans and shellfish. Provided herein is marine peptide of Profile I selected from the group consisting of fish, marine algae, crustaceans, and shellfish. In a particular embodiment, the marine peptide of Profile I is a fish peptide. Isolated herein means for, marine peptide, marine protein hydrolysates generally processed from the marine source more particularly from fish. Isolated is not meant to limit the ability in practicing embodiments herein to combine the isolated ingredients for example with fish or fish products in the preparation of a pharmaceutical product, a dietary supplement or a food.

Still In a further embodiment, provided herein is a unit dose having an effective amount of a marine peptide of Profile I.

Still In a further embodiment, provided herein is a unit dose having an effective amount of a marine peptide of Profile I.

In some embodiments, the animal to be treated can be administered at least one unit dose per day. In some embodiments, the dosage forms can be taken in a single application or multiple applications per day. The term “administering” or “administration” of a composition herein refers to the application of the composition, e.g., oral or parenteral (e.g., transmucosal, intravenous, intramuscular, subcutaneous, rectal, intravaginal, or via inhalation) to the animal. Administering would also include the act of prescribing a composition described herein to an animal by a medical professional. Administering can also include the act of labeling a composition, i.e., instructing an individual or individuals to administer a composition, in a manner as provided herein for treatment. By way of example, administration may be by parenteral, subcutaneous, intravenous (bolus or infusion), intramuscular, or intraperitoneal routes. Dosage forms for these modes of administration may include conventional forms, either as liquid solutions or suspensions, solid forms suitable for solution or suspension in liquid prior to injection, or as emulsions.

In a particularly embodiment a route of administration is oral administration. In a particular embodiment the marine peptide of Profile I is administered to individuals in the form of nutritional supplements, foods, pharmaceutical formulations, or beverages, particularly foods, beverages, or nutritional supplements, more particularly, foods and beverages, more particularly foods. A particular type of food is a functional or medical food (e.g., a food which is in a formulation to be consumed or administered externally under the supervision of a physician and which is intended for the specific dietary management of a disease or condition for which distinctive nutritional requirements, based on recognized scientific principles, are established by medical evaluation).

The marine peptide of Profile I can be formulated in a dosage form. These dosage forms can include, but are not limited to, tablets, capsules, cachets, pellets, pills, gelatin capsules, powders, and granules. Parental dosage forms which include, but are not limited to, solutions, suspensions, emulsions, coated particles, and dry powder comprising an effective amount of the marine peptide of Profile I. In some embodiments, the dosage form can be inserted or mixed into a food substance. Various substances are known in the art to coat particles, including cellulose derivatives, e.g., microcrystalline cellulose, methyl cellulose, carboxymethyl cellulose; polyalkylene glycol derivatives, e.g., polyethylene glycol; talc, starch, methacrylates, etc. In some embodiments, the dosage form is a capsule, wherein the capsule is filled with a solution, suspension, or emulsion comprising a marine peptide of Profile I. It is also known in the art that the active ingredients can be contained in such formulations with pharmaceutically acceptable excipients such as diluents, fillers, disintegrants, binders, lubricants, surfactants, hydrophobic vehicles, water soluble vehicles, emulsifiers, buffers, humectants, moisturizers, solubilizers, preservatives, flavorants, taste-masking agents, sweeteners, and the like. Suitable excipients can include, e.g., vegetable oils (e.g., corn, soy, safflower, sunflower, or canola oil). In some embodiments, the preservative can be an antioxidant, e.g., sodium sulfite, potassium sulfite, metabisulfite, bisulfites, thiosulfates, thioglycerol, thiosorbitol, cysteine hydrochloride, alpha-tocopherol, and combinations thereof.

In one embodiment, a marine peptide of Profile I is provided as a spray dried powder in combination with for example maltodextrin and whey protein wherein the powder comprises about 4%, by weight, of the total weight of the powder of marine peptide of Profile I. The powder is ideally mixed for example in a beverage to provide a dose as described herein.

In one embodiment, the fish protein hydrolysate provided herein have the following ingredient specifications (g/100 g) on a dried weight basis: Protein is provided at about 50-95%, particularly 80-95%, more particularly at about 85-90%, even more particularly at about 88%. Fat is provided at about 0.1% to 10%, particularly at about 0.1%. No carbohydrate. Ash is provided at about 1-15%, particularly at about 5% to about 15%, more particularly at about 10%.

In one embodiment, the degree of hydrolysis _(% DH) is about 5-60%, more particularly greater than 50%.

In one embodiment, the Molecular Weight Range of the peptides (KDa)—are, each independently, on a dried weight basis as follows (Profile 2):

| Peptide (Molecular Weight) | Amount (Dried Weight Basis) |
|---|---|
| >20 KDa | at about <0.1% |
| 15 KDa-20 KDa | at about <0.1 |
| 10 KDa-15 KDa | at about <0.1 |
| 8 KDa-10 KDa | at about 0.1 |
| 6 KDa-8 KDa | at about 0.5% |
| 4 KDa-6 KDa | at about 1.9 |
| 2 KDa-4 KDa | at about 6.3 |
| 1 KDa-2 KDa | at about 13.0 |
| 0.5 KDa-1 KDa | at about 18.5 |
| 0.2 KDa-0.5 KDa | about 23.8 |
| <0.2 KDa | at about 24.0 |
| Free AA | 0-20%, particularly at about 3.5 |
| EAA/NEAA ratio | 0.4-0.9, particularly at about 0.7 |

In one embodiment, the sum of the branched chain amino acid is about 15.31% on a total amino acids basis.

In one embodiment, the indispensable amino acids or essential amino acids is about 36.70% of the total amino acids.

The examples set forth below show that the casein diet supplemented with MP gave approximately a 17% improvement as compared to the control diet, and significantly higher (P<0.05) than the control diet. Preliminary calculations show that the control diet gave a muscle growth in the rat of 5.42 g/day, while the MP-supplemented group gave a significantly higher muscle growth of 6.38 g/day.

In addition to this physiological effect on muscle growth MP leads to a slightly improved utilization of food protein for protein growth.

The examples provided below are not limiting and are for illustrative purposes only.

### EXAMPLES

Experiment 1

A total of 30 group-housed male Wistar rats adapted to the location for 5 days fed a standard rat chow, were evenly weight distributed and transferred to metabolism cages where they were fed 1 of 5experimental diets (n=6 rats per treatment) for 4 days (adaptation period) and allotted 12 g DM/d. Water was provided ad libitum from drinking nipples. The actual feed intake and the weight of the animals recorded. Following the cages were fitted with funnels and tubes for quantitative collection of faeces and urine for 5 days (balance period). The rats were allotted 12 g DM/d and water provided ad libitum. To prevent evaporation of ammonia 1 ml of 20% citric acid was added to the urine collection tubes, covered with gauze, each day. Faeces and urine was collected on daily basis and stored frozen. At the end of the balance period, the rats were weighed, and amount of feed residue, faeces and urine was recorded.

Diets

All diets were designed to contain 20 E % protein, 30 E % fat and 50 E % from carbohydrates (pregelatinized maize starch C-Gel instant™ from Cargill), and contained 5% Vitacel WF 600® cellulose (J. Rettenmaier), 3.5% mineral premix, 1% vitamin premix, 0.25% choline bitartrate, and 0.0014% tert-Butyhyroquinone, included as 9.75% premix to the remainder ingredients. In diet A the protein source was Casein C7078 from Sigma, which was supplemented with 1 DLmethionine from FLUKA. In diet B, C and D marine peptides replaced 20% of casein+methionine on calculated N-basis. In diet C and D NaDNA was added on top of the N coming from casein+methionine and marine peptides. In diet D krill oil (Rimfrost sublime batch no. 10774 60PL) containing 61.5 g PL/100 g partly replaced rape seed oil to maintain a constant lipid content. In diet E all N came from the shrimp roe and may thus compared directly to diet A. Prior to inclusion, the shrimp row was freeze dried and milled, and DM, ash, N and HCl-fat (1) analysed to calculate the inclusion level in the diet.

TABLE 2
Composition of delivered ingredients

| | Dry | | | | | |
|---|---|---|---|---|---|---|
| | matter, | DM | Ash, | N, | Protein, | HCl-fat, |
| | % freeze | factor | % | % | % | % |
| | drying | % | dmb | dmb | dmb | dmb |
| Shrimp roe | 31.01 | 94.53 | 5.48 | 11.04 | 68.99 | 24.02 |
| Marine | | | 97.00 | 14.45 | 14.89 | |
| peptides | | | | | | |

Analyses

Pooled faeces samples (1 per rat) were freeze dried and milled.

Milled diets and faeces samples were analysed for dry matter (to correct for moisture content) and N content based on the Dumas method (2). N in pooled urine samples (1 per rat) was determined by the Kjeldahl method using a Kjeltec 2400.

Calculations

Faecal DM digestibility=(DM intake−Faecal DM output)/DM intake*100

Apparent faecal N digestibility=(N intake−Faecal N output)/N intake*100

N-retention, % of intake=(N intake−Faecal N output−Urinary N output)/N intake*100

N-retention, % of digested=(N intake−Faecal N output−Urinary N output)/(N intake−Faecal N output)*100

Statistics

Data were analyzed by simple one way analysis using GLM in SAS. Results are reported as least square means with their common standard error of mean (SEM), and overall mean. Values with different letters are significantly different, P<0.05. For values where the model is where P<0.15, P-values of significant (P<0.05) and close to significant contrasts (P<0.10) are also reported.

Results

TABLE 3
Body weight at start, end of adaptation
period and end of balance period

| | | Body | Body | Body |
|---|---|---|---|---|
| | | weight, g | weight, | weight, |
| | | Start | g Start | g End |
| | | adaptation | balance | balance |
| Diet kode | Grp | period | period | periode |
| Diet A - Casein | 1 | 116.4 | 132.1 | 159.0 |
| Diet B - Marine peptides (MP) | 2 | 116.4 | 134.3 | 161.8 |
| Diet C - MP + NaDNA | 3 | 116.8 | 135.5 | 160.7 |
| Diet D - MP + NaDNA + PL | 4 | 116.5 | 134.7 | 159.3 |
| Diet E - Shrimp roe | 5 | 116.4 | 133.6 | 159.4 |
| SEM | | 2.6 | 2.5 | 2.3 |
| P-value | | 1.00 | 0.91 | 0.90 |
| Overall mean | | 116.5 | 134.0 | 160.0 |

TABLE 4
Feed and DM intake in adaptation and balance period

| | | Feed intake, g | DM intake, g | | Feed intake, g | DM intake, g | |
|---|---|---|---|---|---|---|---|
| | | adaptation | adaptation | DM intake, | balance | balance | DM intake, |
| Diet kode | Grp | period (4 d) | period (4 d) | g/d | period (5 d) | period (5 d) | g/d |
| Diet A - Casein | 1 | 50.1 | 47.1 | 11.8 | 64.1 | 60.3 | 12.1 |
| Diet B - Marine peptides (MP) | 2 | 50.9 | 48.1 | 12.0 | 64.0 | 60.4 | 12.1 |
| Diet C - MP + NaDNA | 3 | 51.5 | 48.5 | 12.1 | 64.5 | 60.8 | 12.2 |
| Diet D - MP + NaDNA + PL | 4 | 51.8 | 48.9 | 12.2 | 64.8 | 61.1 | 12.2 |
| Diet E - Shrimp roe | 5 | 50.4 | 47.7 | 11.9 | 64.3 | 60.9 | 12.2 |
| SEM | | 0.7 | 0.7 | 0.17 | 0.5 | 0.48 | 0.10 |
| P-value | | 0.44 | 0.43 | 0.43 | 0.79 | 0.70 | 0.70 |
| Overall mean | | 50.9 | 48.1 | 12.0 | 64.4 | 60.7 | 12.1 |

All diets were well accepted well by the rats, as indicated by the uniform feed intake between groups at the target level and uniform growth.

TABLE 5
Faecal excretion and faecal DM digestibility

| | | Faecal | | Faecal | | Faecal DM | | Faecal DM | | DM content in | Faecal DM | |
|---|---|---|---|---|---|---|---|---|---|---|---|---|
| Diet kode | Grp | output, g | | output, g/d | | output, g | | output, g/d | | faeces, % | digestibility, % | |
| Diet A - Casein | 1 | 5.02 | c | 1.00 | c | 3.61 | c | 0.72 | c | 71.93 a | 94.02 | a |
| Diet B - Marine peptides (MP) | 2 | 5.80 | bc | 1.16 | bc | 3.95 | bc | 0.79 | bc | 68.12 b | 93.47 | ab |
| Diet C - MP + NaDNA | 3 | 6.74 | bc | 1.35 | bc | 4.36 | b | 0.87 | b | 64.95 c | 92.83 | bc |
| Diet D - MP + NaDNA + PL | 4 | 6.68 | b | 1.34 | b | 4.21 | b | 0.84 | b | 63.01 c | 93.12 | b |
| Diet E - Shrimp roe | 5 | 9.54 | a | 1.91 | a | 5.00 | a | 1.00 | a | 52.50 d | 91.79 | c |

| | | | | | | |
|---|---|---|---|---|---|---|
| SEM | 0.32 | 0.06 | 0.20 | 0.04 | 1.03 | 0.30 |
| P-value | <0.0001 | <0.0001 | 0.0006 | 0.0006 | <0.0001 | 0.0004 |
| Overall mean | 6.75 | 1.35 | 4.23 | 0.85 | 64.10 | 93.05 |

TABLE 6
N intake, faecal N excretion and apparent faecal N digestibility

| | | | | | | | | | | Apparant |
|---|---|---|---|---|---|---|---|---|---|---|
| | | | | | | Faecal N | | Faecal N | | faecal N |
| Diet kode | Grp | N intake, g | | N intake, g/d | | output, g | | output, g/d | | digestibility, % |
| Diet A - Casein | 1 | 1.866 | d | 0.373 | d | 0.0920 | c | 0.0184 | c | 95.20 |
| Diet B - Marine peptides (MP) | 2 | 2.136 | b | 0.427 | b | 0.1093 | bc | 0.0219 | bc | 94.99 |
| Diet C - MP + NaDNA | 3 | 2.036 | c | 0.407 | c | 0.1189 | ab | 0.0238 | ab | 94.26 |
| Diet D - MP + NaDNA + PL | 4 | 2.169 | ab | 0.434 | ab | 0.1152 | b | 0.0230 | b | 94.79 |
| Diet E - Shrimp roe | 5 | 2.197 | a | 0.439 | a | 0.1382 | a | 0.0276 | a | 93.82 |

| | | | | | |
|---|---|---|---|---|---|
| SEM | 0.016 | 0.003 | 0.0076 | 0.0015 | 0.36 |
| P-value | <0.0001 | <0.0001 | 0.0054 | 0.0054 | 0.07 |
| Overall mean | 2.081 | 0.416 | 0.1147 | 0.0229 | 94.61 |

1 vs 5: P = 0.011
1 vs 3: P = 0.074
2 vs 5: P = 0.028

The N content in the casein+methionine mix appear to have been overestimated when mixing the diets, which explains the slightly lower N intake in group A.

TABLE 7
Urinary excretion

| | | Urinary | Urinary | Urinary N | Urinary N |
|---|---|---|---|---|---|
| Diet kode | Grp | output, g | output, g/d | output, g | output, g/d |
| Diet A - Casein | 1 | 41.4 c | 8.3 c | 0.849 b | 0.170 b |
| Diet B - Marine peptides (MP) | 2 | 61.7 b | 12.3 b | 0.933 ab | 0.187 ab |
| Diet C - MP + NaDNA | 3 | 51.8 bc | 10.4 bc | 1.009 a | 0.202 a |
| Diet D - MP + NaDNA + PL | 4 | 57.2 bc | 11.4 bc | 1.034 a | 0.207 a |
| Diet E - Shrimp roe | 5 | 86.3 a | 17.3 a | 1.050 a | 0.210 a |
| SEM | | 6.8 | 6.8 | 0.041 | 0.008 |
| P-value | | 0.0015 | 0.0015 | 0.011 | 0.011 |
| Overall mean | | 59.7 | 11.9 | 0.975 | 0.195 |

TABLE 8
N retention in grams, g/d, % of intake and % of digested N

| | | | | | |
|---|---|---|---|---|---|
| Diet A - Casein | 1 | 0.967 bc | 0.193 bc | 50.73 | 53.31 |
| Diet B - Marine peptides | 2 | 1.135 a | 0.227 a | 52.25 | 54.96 |
| (MP) | | | | | |
| Diet C - MP + NaDNA | 3 | 0.944 c | 0.189 c | 45.57 | 48.35 |
| Diet D - MP + NaDNA + PL | 4 | 1.062 ab | 0.212 ab | 48.03 | 50.66 |
| Diet E - Shrimp roe | 5 | 1.045 abc | 0.209 abc | 46.79 | 49.89 |
| SEM | | 0.037 | 0.007 | 1.86 | 1.93 |
| P-value | | 0.008 | 0.009 | 0.10 | 0.14 |
| Overall mean | | 1.031 | 0.206 | 48.67 | 51.43 |

2 vs 3: P = 0.018
2 vs 5: P = 0.049
1 vs 3: P = 0.061
2 vs 3: P = 0.023
2 vs 5: P = 0.075
1 vs 3: P = 0.081

It was assumed that nitrogen retention is equal to protein retention, which is equal to protein accretion, of which 90% is equal to muscle accretion. Therefore any increase in protein retention is therefore therefore mainly due to muscle growth.

The study shows that the control diet with casein as the sole source of protein gave a nitrogen retention of 0.193 g/day while the casein diet supplemented with MP gave a nitrogen retention of 0.227 g/day, ≈17% improvement as compared to the control diet, and significantly higher (P<0.05) than the control diet. The N retention in terms of digested and absorbed N was 53.31 and 54.96%, respectively, equal to 3.1% better utilisation of dietary protein if supplemented with MP as compared to the control diet. Using the factor N×6.25 and assuming that protein content in muscle is 20%, and that 90% of the nitrogen retention is in muscle tissue, preliminary calculations show that the control diet gave a muscle growth in the rat of 5.42 g/day, while the MP-supplemented group gave a significantly higher muscle growth of 6.38 g/day.

In addition to this physiological effect on muscle growth MP leads to a slightly improved utilization of food protein for protein growth.

1. A method of increasing muscle growth and recovery in an animal comprising administering to the animal in need of muscle growth or in need of such recovery an effective amount of a marine peptide of Profile 2:
Profile [[I]]2

| Peptide (Molecular Weight (Da) | Amount (Dried Weight Bases) |
|---|---|
| >20 KDa | less than 0.1% |
| 15 KDa-20 KDa | less than 0.1% |
| 10 KDa-15 KDa | less than 0.1% |
| 8 KDa-10 KDa | 0.1% |
| 6 KDa-8 KDa | less than 0.5% |
| 4 KDa-6 KDa | 1.9% |
| 2 KDa-4 KDa | 6.3% |
| 1 KDa-2 KDa | 13% |
| 0.5 KDa-1 KDa | 18.5% |
| 0.2 KDa-0.5 KDa | 23.8% |
| <0.2 KDa | 24.8% |
| Free AA | 0-20% |
| EAA/NEAA ratio | 0.4-0.9% |

2. The method as recited in claim 1 wherein the animal is a mammal.

3. The method as recited in claim 2 wherein the mammal is a human.

4. The method as recited in claim 1, wherein the amount of marine peptide administered is from about 40 mg of the peptide per one Kg of body weight of the animal.

5. The method as recited in claim 1, wherein the amount of marine peptide administered is about 20 mg of the marine peptide per one Kg of body weight of the animal.

6. The method as recited in claim 3 wherein the amount delivered to the human is from about 1 gram up to about 10 grams.

7. The method as recited in claim 6 wherein the amount delivered to the human is from about 2 grams up to about 10 grams.

8. The method as recited in claim 7 wherein the amount delivered to the human is from about 2 gram up to about 5 grams.

9. The method as recited in claim 8 wherein the amount delivered to the human is from about 2 gram up to about 3 grams.

10. The method as recited in claim 1 wherein the amount of the Free AA is 3.5%.

11. The method as recited in claim 1 wherein the EAA/NEAA ratio is 0.7.
